(19) United States
(12) Patent Application Publication
(10) Pub. No.: US 2024/0024387 A1
(43) Pub. Date: Jan. 25, 2024

(54) PROBIOTIC DELIVERY SYSTEMS AND METHODS OF MAKING AND USING

(71) Applicant: University of Louisville Research Foundation, Inc., Louisville, KY (US)

(72) Inventors: Jill M. STEINBACH-RANKINS, Crestwood, KY (US); Donald R. DEMUTH, Louisville, KY (US)

(21) Appl. No.: 18/248,491

(22) PCT Filed: Oct. 15, 2021
(86) PCT No.: PCT/US2021/055257

§ 371 Date: Apr. 10, 2023

Related U.S. Application Data
(60) Provisional application No. 63/091,956, filed on Oct. 15, 2020.
(60) Provisional application No. 63/156,822, filed on Mar. 4, 2021.

(51) Int. Cl. A61K 35/747; B33Y 10/00; A61K 35/745; B33Y 70/10; A61K 9/20; A61P 31/04

(52) U.S. Cl. CPC A61K 35/747; B33Y 10/00; A61K 35/745; B33Y 70/10; A61K 9/2095; A61K 9/205; A61P 31/04; C12R 2001/23

(57) ABSTRACT

A probiotic delivery system is provided, as are methods of making and using such a probiotic delivery system.

### CROSS REFERENCE TO RELATED APPLICATIONS

This application claims the benefit of priority under 35 U.S.C. 119(e) to U.S. Application No. 63/091,956 filed on Oct. 15, 2020, and U.S. Application No. 63/156,822 filed on Mar. 4, 2021, the entirety of which are incorporated herein by reference.

### FEDERALLY SPONSORED RESEARCH OR DEVELOPMENT

This invention was made with government support under DE025345, DE014605 and AI139671 awarded by the National Institutes of Health (NIH). The government has certain rights in the invention.

### TECHNICAL FIELD

This disclosure generally relates to probiotics and delivery of probiotics.

### BACKGROUND

Probiotics are considered “beneficial bacteria”, and can be used to reduce or prevent the detrimental effects caused by infectious or pathogenic bacteria. Hence, probiotics can provide a potential therapeutic avenue.

Currently, however, there are limited numbers of delivery vehicles available for localized and sustained release of probiotics for therapeutic purposes. Thus, delivery vehicles that provide for sustained and prolonged release of probiotics are needed.

### SUMMARY

A probiotic delivery system is described herein, as well as making and using such a probiotic delivery system.

In one aspect, methods of making a probiotic delivery system are provided. Such methods typically include the steps of: combining at least one probiotic with bioink to produce a probiotic-seeded bioink; and printing a three-dimensional structure using the probiotic-seeded bioink.

In some embodiments, the at least one probiotic is a Lactobacillus probiotic, a Bifidobacterium probiotic, or a combination thereof. Representative probiotics from the Lactobacillus genus include, without limitation, L. acidophilus, L. crispatus, L. rhamnosus, L. gasseri, L. reuteri, L. bulgaricus, L. plantarum, L. johnsonii, L. paracasei, L. casei, and L. salivaris. Representative probiotics from the Bifidobacterium genus include, without limitation, B. bifidum, B. longum, B. breve, B. infantis, B. lactis, and B. adolescentis.

In some embodiments, the bioink includes agarose, alginate, chitosan, collagen, decellularized extracellular matrix (ECM), fibrin/fibrinogen, gelatin, graphene, hyaluronic acid (HA), hydroxyapatite, PCL/PLA/PLGA, silicone, Pluronic F127, polyethylene glycol/oxide, or combinations thereof.

In some embodiments, the bioink comprises alginate and gelatin. In some embodiments, the bioink comprises about 2% w/v alginate and about 10% w/v gelatin.

In some embodiments, between about 10\hat{ }6 and 10\hat{ }9 cfu per mg polymer (e.g., 10\hat{ }7 to 10\hat{ }8 CFU per mg polymer) of the probiotic are added to the bioink.

In some embodiments, the printing includes extrusion and co-axial extrusion, fused deposition modelling, inkjet bio-printing, laser-assisted bioprinting, stereolithography, selective laser sintering (SLS), or combinations thereof.

In some embodiments, the structure is line-shaped, cylindrical-shaped, disc-shaped, spherical-shaped, oval-shaped, cube-shaped, layered, a structure with discreet compartments, or combinations thereof.

In some embodiments, the methods described herein further include crosslinking the scaffold. Representative methods of crosslinking include applying genipin, calcium chloride, gelain methacryloyl, or combinations thereof.

In one aspect, probiotic delivery systems made by the method described herein are provided. In another aspect, probiotic delivery systems are provided that include a bioink and a probiotic.

In still another aspect, methods of treating a bacterial infection in an individual are provided. Such methods typically include administering a probiotic delivery system as described herein to the individual.

In yet another aspect, methods of treating a bacterial infection in an individual are provided. Such methods typically include 3D printing a probiotic delivery system as described herein; and administering the 3D printed probiotic delivery system to an individual. For example, representative bacterial infections can be caused by the presence of at least Porphyromonas gingivalis or Gardnerella vaginalis.

The compositions and methods described herein are advantageous over current technologies such as electrospun fibers and intravaginal rings because of the rapid synthesis and shape specificity provided by 3D bioprinting. Moreover, the concept of a platform incorporating probiotics for sustained delivery within a body cavity (e.g., the oral cavity, the vaginal cavity) is novel.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 are photographs of exemplary shapes of scaffolds as described herein. Scale bar on left, 1 mm; scale bar on right, 0.5 mm.

FIG. 2 are photographs showing exemplary structures used to measure the release of a probiotic from a scaffold as described herein.

FIG. 3 is a graph showing probiotic loading of a scaffold as described herein.

FIG. 4 is a graph showing the rate of release of a probiotic from a scaffold as described in FIG. 2.

FIG. 5 is a graph showing the dose-dependent inhibition of P. gingivalis (P.g.) adhesion to TIGK cells after pre- or simultaneous application of L. acidophilus (L.a.) at different MOIs. The binding of P.g. or L.a. alone to TIGK cells is represented by black and gray histograms respectively. The inhibition of P.g. binding after simultaneous or pre-application of L.a. is shown by striped histograms. Error bars represent the standard deviation of the mean CFU value.

FIG. 6A shows the macrostructure of bioprinted rings loaded with the 5×10⁷ CFU/mg of L.cr. and crosslinked with both CaCl₂ and genipin for 24 hr.

FIG. 6B shows a scanning electron microscopy image of probiotic scaffold cross-section after 1 wk in MRS. SEM scale bar represents 5 μm.

FIG. 7 is a graph showing the cumulative release and proliferation of L.cr. from bioprinted scaffolds loaded with 5×10⁷ CFU/mg (as described in FIG. 6A-6B) over 4 wk in different media. Release values are shown as the mean±standard deviation of eluates from three independent ring scaffolds and, in some cases, error bars are smaller than the symbol size.

FIG. 8A-8F are graphs that show probiotic binding to TIGK cells observed after administration of different probiotic MOIs (FIG. 8A-8C) and inhibition of P. gingivalis adhesion after different doses of probiotic pre- and co-treatment (FIG. 8D-8F). Statistical significance between P. gingivalis alone and probiotic-treated groups was calculated by one-way ANOVA and is represented by *P≤0.05. **P≤0.01, and ***P≤0.001.

FIG. 9 is a graph showing that TIGK cells exhibit a dose-dependent increase in P. gingivalis internalization. Values represent the mean±standard deviation of P. gingivalis in CFU/mL. Statistical significance in internalization as a function of administered P.g. dose was calculated by one-way ANOVA and is represented by ****P≤0.0001.

FIG. 10 is a graph showing probiotic viability determined pre- and post-printing after initial incorporation of 5×10⁷ CFU L.a. per mg scaffold. Values represent the mean±standard deviation of post-print viability. Statistical significance was calculated by t-test and is represented by ***P≤0.001.

FIG. 11 is a graph showing cumulative release of L.a. from 3D-bioprinted scaffolds in PBS, MRS, and artificial saliva. Values represent the mean±standard deviation of cumulative release at each time point. Statistical significance was calculated by t-test and is represented by p≤0.05. **Data in collaboration with Veeresh Rai.

FIG. 12 are graphs showing the results of the print resolution assay.

FIG. 13 show the results of post-print viability and probiotic release and proliferation.

FIG. 14 show the proliferation and morphology of L.cr.-containing scaffolds over time.

FIG. 15 are graphs that show the effect of cross-linking on scaffold mass loss and swelling.

FIG. 16 are graphs that show the viability of vaginal keratinocytes.

### DETAILED DESCRIPTION

3D bioprinting is a relatively new technique that can be used for rapid prototyping in numerous applications. In 3D bioprinting, a polymer solution referred to as a bioink is used to create a scaffold with a complex three-dimensional geometry. These solutions often contain cells or biomaterials that mimic extracellular matrix and support cellular adhesion, proliferation, and differentiation.

Here, 3D bioprinted scaffolds loaded with a probiotic are described as well as their ability to provide sustained release of beneficial bacteria into a surrounding environment. The compositions described herein can inhibit bacterial infections such as, without limitation, Porphyromonas gingivalis, a bacteria involved in oral biofilms, or Gardnerella vaginalis, a bacteria implicated in vaginosis infections.

### Probiotics

The World Health Organization (WHO) has defined probiotics as “live micro-organisms, which when administered in adequate amounts, confer a health benefit to the host.” Among a number of probiotics, Lactobacillus and Bifidobacterium genera have shown promising results in inhibiting bacterial infections (e.g., due to P. gingivalis or G. vaginalis). Therefore, any number of Lactobacillus spp., Bifidobacterium spp., or combinations thereof can be used as described herein.

Representative Lactobacillus species include, without limitation, L. acidophilus, L. rhamnosus, L. gasseri, L. reuteri, L. bulgaricus, L. plantarum, L. johnsonii, L. paracasei, L. casei, L. crispatus, and L. salivaris. Representative Bifidobacterium species include, without limitation, B. bifidum, B. longum, B. breve, B. infantis, B. lactis, and B. adolescentis.

Typically, a probiotic from a liquid or lyophilized culture can be used to print a scaffold as described herein. A probiotic in a liquid culture can be from a primary culture, or the probiotic in a liquid culture can originate from a frozen or lyophilized state. Typically, a probiotic in log phase growth would be used in the methods described herein to print a scaffold. In some embodiments, about 10\hat{ }6 and 10\hat{ }9 cfu per mg polymer (e.g., 10\hat{ }7 to 10\hat{ }8 CFU per mg polymer). A suitable number of probiotic bacteria should be used that maintains the print line resolution and integrity, while achieving maximum post-printing viability.

### Bioinks and 3D Bioprinting

3D bio-printing techniques provide a valuable platform because of their ability to precisely pattern living cells into biocompatible materials in a pre-defined manner. 3D bio-printing can be broken down into three basic stages: the pre-printing stage, the printing stage, and the post-printing stage. The pre-printing stage include, without limitation, bioink preparation, cell culture, and model preparation for printing; the printing stage includes, without limitation, printing the scaffold and cross-linking the scaffold, and the post-printing stage includes, without limitation, crosslinking and maintenance of the scaffold.

Bioinks generally are a combination of one or more polymers. The polymers used in bioinks typically are chosen based on their specific properties. There are a number of different types of bioinks. For example, there are matrix bioinks, which are cell-laden mixtures used to shield the cells from the shear stress of printing, sacrificial bioinks, which are materials that can be removed after printing, and support bioinks, which are materials with specific mechanical properties. Examples of matrix bioinks include, without limitation, collagen, fibrin, gelatin, silk, cell or tissue derived ECM, alginate, polyethylene glycol/oxide, or agarose; examples of sacrificial bioinks include, without limitation, gelatin, agarose, pluronic, or carbohydrate glass; and examples of support bioinks include, without limitation, polylactic acid, poly(L-lactic acid), poly(lactic-co-glycolic acid), polycaprolactone, or silicone.

One combination that was shown to work particularly well is a combination of alginate (e.g., sodium alginate) and gelatin. Gelatin and alginate both are attractive for use in 3D bioprinting because they are biocompatible and bioinert. Additionally, gelatin provides structural maintenance, and the chemical crosslinking of alginate is mild in nature. Bioinks that include gelatin and alginate have demonstrated printability, cell viability, proliferation, adhesion, and release of cell specific markers within the scaffold. Alternatively, a number of other natural biomaterials such as, for example, agarose and fibrin based bioink, can be 3D printed and used to deliver the probiotics as described herein.

Bioinks can include about 1.0% to about 3% w/v alginate and about 5% to about 15% w/v gelatin (e.g., about 1.5% to about 2.5% w/v alginate and about 7.5% to about 12.5% w/v gelatin; about 2% w/v alginate and about 10% w/v gelatin).

Representative, non-limiting shapes of the structures described herein include line-shaped, cylindrical-shaped, disc-shaped, spherical-shaped, oval-shaped, cube-shaped, or combinations thereof.

With respect to the printing stage, there are a number of 3D printing techniques that are suitable and can be used to fabricate (“biofabricate”) the compositions described herein (e.g., processes that don't require chemical solvents or high temperatures). 3D printing techniques include, for example, extrusion and co-axial extrusion, fused deposition modelling, inkjet bio-printing, laser-assisted bioprinting, stereolithography, selective laser sintering (SLS), or combinations thereof. It would be appreciated that essentially any 3D bioprinting process can be used that allows for the ability to print an aqueous solution at a temperature and pressure that is not harmful to the cells.

Post-printing, the scaffold can be cross-linked using, for example, genipin, calcium chloride, or gelatin methacryloyl (GelMA) accompanied by UV crosslinking, if desired. Additionally or alternatively, the scaffold (crosslinked or not crosslinked) can be evaluated for printability, degradation rate, bacterial viability and loading, release kinetics, pH alterations to surrounding environment, and/or lactic acid production using techniques described herein and/or known in the art.

3D bioprinting has a number of advantages over other techniques. For example, the shape can be customized and the methods allow for the freedom of creating complex architecture using a wide variety of materials. The methods described herein allow for rapid and precise spatial arrangement and distribution of cells compared to traditional tissue engineering methods, and also are less time consuming relative to molding or inverse molding techniques. Using the 3D bioprinting methods described herein, high throughput production of scaffold structures can be achieved.

See, for example, 3D Bioprinting: Bioink Selection Guide, 2019, retrieved from sigmaaldrich.com/technical-documents/articles/materials-science/3d-bioprinting-bioinks.html on the World Wide Web.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—Bioink and Bioprinting

Scaffolds were printed using gelatin, alginate and Lactobacillus crispatus to evaluate the release kinetics of the bacteria, degradation rates of the scaffolds with and without bacteria, lactic acid production from the bacteria, pH changes resulting from bacteria proliferation and lactic acid production, and efficacy of the construct in reducing G. vaginalis infection in soluble and epithelial co-culture assays. In addition, new architectures for oral bacteria delivery are described.

Bioink Preparation

Several different ratios of gelatin to alginate were tested, as most literature sources utilized 10-20% w/v gelatin and 1-5% w/v alginate. The bioinks for testing were comprised of gelatin from bovine skin, type B (Sigma, MO) and sodium alginate (MP Biomedicals, LLC, OH). Gelatin and sodium alginate were dissolved in MRS broth (Sigma-Aldrich, MO) in the ratios of 10:1, 10:2, 11:2, 12:2, and 16:4 w/v, followed by overnight incubation at 37° C.

Bacterial Isolation

Lactobacillus crispatus, Strain MV-1A-US (L.cr) (American Type Culture Collection (ATCC), CA) stock solution was kept frozen at −60° C. until use. Stock solution was diluted and streaked on MRS agar (Sigma-Aldrich, MO) plates. L.cr. was then cultured in MRS broth and used between passages three and five. To measure OD600, 1:10 dilution of bacteria solution was made by diluting 100 μL of bacteria in MRS broth with 900 μL of PBS, and the absorbance was read using a Nanodrop 2000 (Thermo Scientific, MA).

Based on the absorbance, the volume of L.cr. in MRS broth needed was determined per mg of polymer used in the ink. The determined volume was centrifuged at 3,500 g for 10 minutes. The supernatant was discarded, and the pellet was resuspended in 500 μL of MRS. The bacteria were then transferred to the prepared bioink, and the mixture was vortexed. After vortexing, the bioink was transferred to a syringe and incubated at 37° C. for 15 minutes.

3D Bioprinting of Constructs

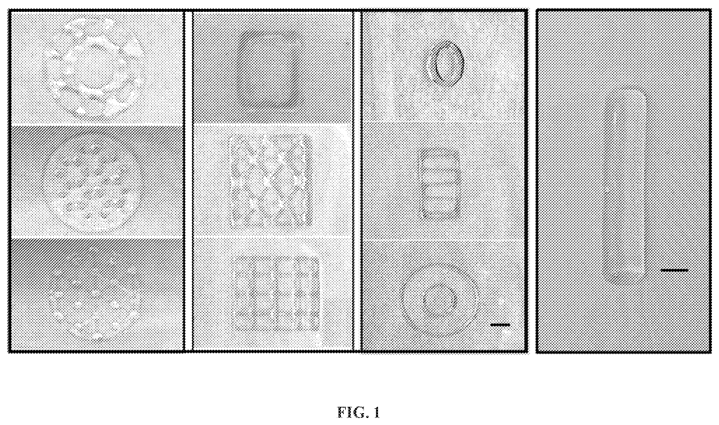

The syringe of bioink was transferred to the extruder of the Allevi 3 Bioprinter (Allevi, PA), and a 30-gauge luer lock needle was attached to the end of the syringe. The Allevi 3 was calibrated, and optimized parameters were set for printing. The scaffold was printed based on a pre-made GCODE design. See FIG. 1. After printing, scaffolds were transferred to 4° C. for 10 minutes.

A 10% w/v calcium chloride (CaCl₂) solution was made from calcium chloride hexahydrate (Acros Organics, NJ) for crosslinking. The CaCl₂ and deionized (DI) water were chilled at 4° C. After the scaffolds were refrigerated for 10 minutes, they were crosslinked with 5 mL 10% w/v CaCl₂ and placed back at 4° C. for 5 minutes. The scaffolds were then washed three times with 3 mL DI water. After crosslinking, samples were ready for testing.

### Example 2—Efficacy of Scaffold

Viability Test and Degradation Assay

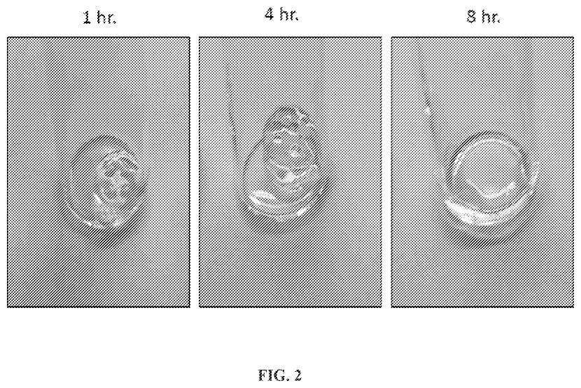

The temperature and pressure of the printer during scaffold preparation can affect the viability of the probiotic. Therefore, the ratio of the bacteria that survived following printing and the amount of bacteria loaded in the scaffold was used to determine viability. Degradation was determined by measuring changes in the weight of a scaffold at different time points. See FIG. 2.

Loading and Release Test

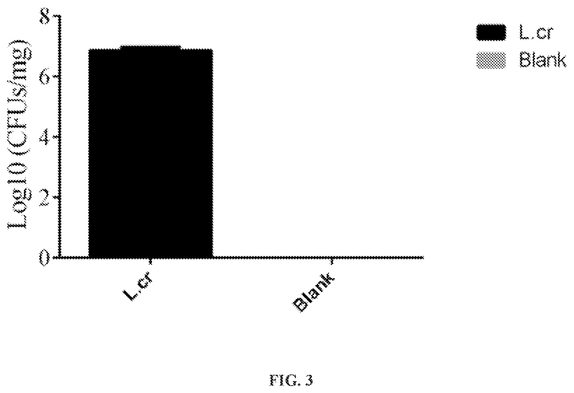

After printing, un-crosslinked blank and bacterial samples were evaluated for loading by dissolving pre-weighed samples in 5 mL broth in Eppendorf tubes at 37° C. for 1 hour. See FIG. 3.

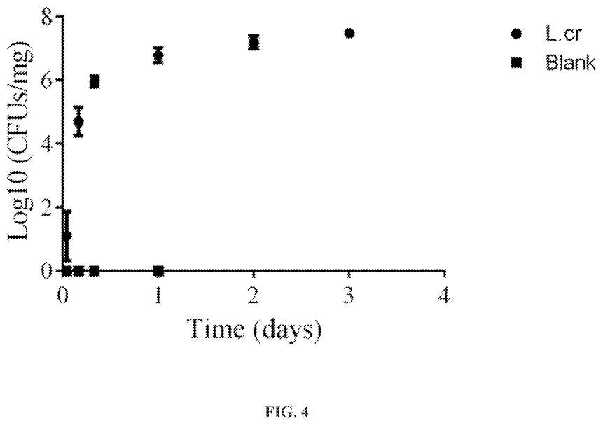

Release of bacteria was evaluated by incorporating scaffold in MRS broth and counting the CFU in the bacteria. Printed and cross-linked scaffolds were weighed and placed in 5 mL tubes, and 5 mL MRS broth was added for time points of 1, 4, 8, 24, 48, 72 hours, 1 week, and 2 weeks. For each sample, the supernatant was then serially diluted by adding 204 of sample to 1804 of MRS broth, and 2 μL of samples were plated on MRS agar plates. The plates were placed in the anaerobic chamber for two days and evaluated for colony-forming units (CFU) counts. See FIG. 4.

The ability of the scaffolds described herein to inhibit P. gingivalis is assessed using adhesion assays, antibiotic protection assays, and ELISAs. Adhesion assays are used to measure the amount of labeled P. gingivalis bound to TIGKs using a Spectra Max cell analyzer. Antibiotic protection assays are used to evaluate the ability of P. gingivalis to invade cells (e.g., TIGK cells) and are performed by challenging TIGKs with P. gingivalis, treating the cells with antibiotics to kill external bacteria, lysing the TIGKs followed by plating and counting the internalized CFUs. ELISAs are performed in triplicate to evaluate changes in TNF and IL-8 levels, and statistical significance among different probiotics groups is measured by one-way ANOVA test.

### Example 3—Adhesion Assay

An adhesion assay was performed to determine the ability of L. acidophilus (L.a.) to adhere to telomerase immortalized gingival keratinocyte (TIGK) cells to prevent Porphyromonas gingivalis (P.g.) adhesion. The adhesion assay was conducted with three groups: negative control (TIGK cells only), positive control (TIGKs individually treated by either P.g. or L.a.), and two experimental groups in which P.g. and L.a. were either applied together to TIGK cells, or TIGKs were pre-treated with L.a. for 15 min prior to P.g. addition.

Briefly, TIGKs were plated in 24-well plates at a density of 1×10\hat{ }5 per well and incubated for 2 days before treating with bacteria. After 2 day incubation, cells were treated with 500 μL of carboxyfluorescein succinimidyl ester (CS)-labeled P.g. (ATCC-33277) at a multiplicity of infection (MOI) of 2000 and L.a. (ATCC-4356) at different MOIs (100, 500, 1000, 2000) to evaluate the effect of L.a. dose on P.g. adhesion to TIGK cells.

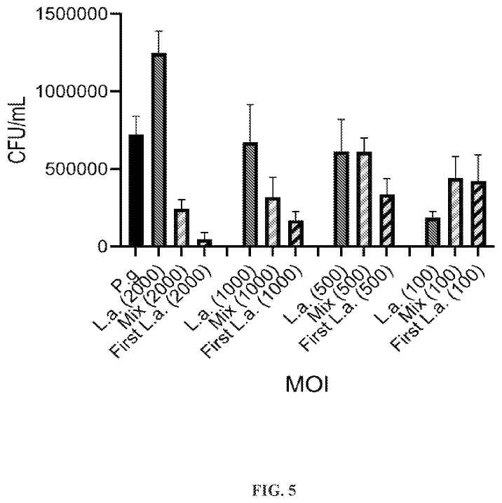

In the first experimental group, fluorescently-labeled P.g. (500 μL, MOI of 2000) and unlabeled L.a. (500 μL, MOI 100, 500, 1000, 2000) were applied at the same time (for 90 min total). For the second experimental group, TIGKs were treated with L.a. (500 μL) at different MOIs (100, 500, 1000, 2000) for 15 min, followed by the application of fluorescently-labeled P.g. (500 μL) at an MOI of 2000 for an additional 75 min to allow bacterial adhesion to TIGKs. After incubation, the supernatant was removed and TIGKs were washed three times with phosphate buffered saline (PBS). The fluorescence of adhered bacteria was measured using a SpectraMax microplate reader at wavelengths of 485/535 nm (excitation/emission) for L.a. or P.g. Fluorescence curves for P.g. and L.a. were prepared by serially diluting 2×10\hat{ }8 fluorescently-labeled P.g. and L.a. bacterial cells and measuring fluorescence at different dilutions. Adhered bacterial CFU count was determined by converting fluorescence to CFUs with the help of a standard fluorescent curve for each of P.g. and L.a. FIG. 5.

### Example 4—Preliminary Results

3D printed scaffolds (e.g., containing 10% gelatin and 2% alginate) initially possess about 70% cell viability, sustainably deliver probiotics for a prolonged duration (e.g., about 14 days), and reduce adhesion and invasion of P. gingivalis to TIGKs. Such 3D-printed scaffolds also modulate pro-inflammatory cytokine (e.g., TNF and IL-8) levels. Thus, these experiments demonstrate that 3D printed scaffolds containing probiotics can be utilized as a localized delivery system for mitigating the effects of P. gingivalis.

### Example 5—Development of 3D-Bioprinted Scaffolds Containing Probiotics for Oral Delivery Applications

Objectives: Porphyromonas gingivalis (P. gingivalis), a key pathogen in periodontitis, adheres to and invades gingival epithelial cells, resulting in decreased cell viability. Previous studies have indicated that probiotic organisms are effective against many dental pathogens; however, few approaches provide sustained-delivery of active agents in the oral cavity. Three-dimensional (3D)-bioprinting presents a novel approach to orally deliver probiotics, by fabricating well-defined cell-laden architectures and modulating active agent release. A variety of biopolymers have been investigated as “bioinks” and gelatin-alginate, in particular, is a promising candidate due to its structural stability, host-immune compatibility, and viable probiotic incorporation. In this work, we first evaluated free probiotics to reduce P. gingivalis adhesion and invasion to telomerase immortalized gingival keratinocytes (TIGKs). In parallel, we sought to establish preliminary proof-of-concept of 3D-bioprinted scaffolds to sustain probiotic delivery (>1 week), and reduce P. gingivalis adhesion to TIGKs.

Methods: Lactobacillus reuteri (“L.r.”), Lactobacillus acidophilus (“L.r.”), and Bifidobacterium bifidum were adminstered to P. gingivalis-treated TIGKs. The inhibition of P. gingivalis (ATCC 33277) adhesion to, and invasion of, TIGKs after probiotic treatment was measured by cell-bound fluorescence and antibiotic protection assays, respectively. Probiotic scaffolds containing ˜5×10⁷ CFU/mg were assessed for degradation, and release over one week and were similarly evaluated for adhesion inhibition. Statistical significance in treatment efficacy between different probiotic groups was determined by one-way ANOVA.

Results: Free L.a., L.r., and B. bifidum administration improved the viability of TIGKs by reducing adhesion (and invasion) of P. gingivalis to TIGKs by 90%, 80%, and 95%, respectively. Additionally, novel probiotic-containing scaffolds were successfully produced, demonstrating high viability and sustained-release of ˜10⁷ to 10⁸ CFU/mg daily over one week, with similar reductions in adhesion.

Conclusion & Practical Implications: Probiotics effectively limit P. gingivalis adhesion and invasion to TIGKs, suggesting that 3D-bioprinted probiotic-containing scaffolds is a novel and promising localized and sustained-delivery system for mitigating P. gingivalis effects in periodontitis.

### Example 6—Bioprinting: Gelatin-Alginate Ink Formulation and Procedure

### Preparation of Bioink and Incorporation of Bacteria

The bioinks were comprised of sodium alginate (Sigma Aldrich, prod #W201502) and gelatin from bovine skin (Sigma Aldrich, prod #G9391). Sodium alginate and gelatin were dissolved in De Man, Ragosa, and Sharpe (MRS) broth at a 10:2 ratio (10% w/v gelatin to 2% w/v sodium alginate), followed by overnight incubation at 37° C. To incorporate L.cr. in the bioprinted scaffolds, L.cr. were subcultured in MRS broth (15-18 hr). After subculture, L.cr. was diluted 1:10 in PBS and the optical density at 600 nm (OD₆₀₀, Nanodrop 2000, Thermo Scientific, MA, USA) was measured to determine the volume of L.cr. to incorporate in the bioink (calculated as CFU/mg polymer). The corresponding volume of L.cr. solution was then centrifuged (3500×g, 10 min), followed by removal of the supernatant and resuspension in 300 μL of MRS broth. L.cr. solution was then transferred to 1.7 mL of prepared sodium alginate-gelatin bioink and the mixture was vortexed. The bioink transferred to a syringe for bioprinting and incubated at 37° C. for 5 min in the printer cartridge.

### Bioprinting and Crosslinking of the Scaffolds

An Allevi 3 Bioprinter (Allevi, PA, USA) was used to bioprint the scaffolds. The 3D printer was calibrated, and processing parameters such as extruder temperature and pressure, printing speed, and bed temperature were optimized for printing. The extruder temperature and pressure were adjusted within the range of 34-37° C., and 32-42 psi. The syringe of bioink was placed into the extruder and 26, 30, or 34-gauge luer lock needles were attached to the end of the syringe. The scaffold design was extracted from gcode provided by Allevi onto the GUI for Allevi Bioprint Essential. Printed ring-shaped scaffolds (OD=4 mm, ID=3 mm) were then placed in a refrigerator (4° C.) for 15 min. Different crosslinking reagents including genipin (covalent crosslinking of gelatin) and CaCl₂ (ionic crosslinking of alginate) were assessed to determine the resulting structural stability of the bioprinted gelatin-alginate scaffold. For scaffolds crosslinked with CaCl₂ alone, 10 mL of CaCl₂ in DI water (10% w/v) were poured on the chilled scaffolds in a petri dish, followed by incubating the petri dish at 4° C. for 15 min. Crosslinked scaffolds were then washed with DI water three times and returned to the freezer until use. Similarly, for gelatin-only crosslinked scaffolds, a 0.5 wt % genipin (or 0.502% w/v) solution in 1×PBS was used to crosslink the gelatin. Genipin solution was poured on the chilled scaffolds in a 5 mL scintillation vial and incubated at room temperature for 24 hr. Crosslinked scaffolds were then washed with 1×PBS three times and returned to the freezer (−20° C.) for long-term storage. For dual-crosslinked scaffolds, scaffolds were crosslinked first with CaCl₂ and subsequently with genipin. Scaffolds were similarly reacted with CaCl₂ (15 min, 4° C.), washed three times with 3 mL DI water, then crosslinked with 5 mL 0.5 wt % genipin solution for 24 hr at room temperature. The scaffolds were then washed three times with 3 mL 1×PBS and placed in the freezer for long-term storage.

### Example 7—Silicone Printing

### Bioink Preparation

A mixture of vinyl terminated polydimethylsiloxane (70%) and vinyl, methyl modified silica (30%) (“part A”) and methyl hydrosiloxane-dimethylsiloxane copolymer, trimethylsiloxane-terminated (“part B”) was purchased from Allevi, Inc. (Allevi, PA, United States) and mixed in a 10:1 w/w ratio (part A:part B, 1.0 g part A, 0.1 g part B). Metronidazole (Sigma Aldrich, M3761-5G) was ground into a fine powder using a mortar and pestle, and 55 mg of metronidazole was transferred to a 5 mL eppendorf tube to formulate the metronidazole-containing bioink. Metronidazole was dissolved in a minimum volume of 60 μL DMSO (Fischer Scientific, B231-1), added to the eppendorf, and vortexed to obtain a homogeneous suspension. The bioink was then mixed and added to the metronidazole suspension, stirred with a spatula for 5 min, and subsequently transferred to a 3 mL syringe for bioprinting. The resulting bioink contains 50 μg metronidazole/mg scaffold and a final DMSO concentration of 5.4 wt %.

### Bioprinting

A 23-gauge needle was attached to the syringe and connected to the CORE head of the bioprinter. The scaffold design was developed in SOLIDWORKS®, and was extracted as an .stl file to the GUI for Allevi Bioprint Essential. The scaffold design consisted of a hollow cylindrical shape (OD=4 mm, ID=2 mm, height=5 mm, shell thickness=0.25 mm). The printing design parameters were set to obtain: layer thickness (0.2 mm), extrusion rate (5 mm/sec), infill grid design (zig zag), and infill grid (0.5 mm). Next, the parameters for extruder were set at 30° C. and 100 psi during the printing for consistent extrusion. The final bioprinted scaffolds resulted in OD=4.07±0.02 mm, ID=2.43±0.32 mm, and height=4.97±0.15 mm with a mass of ±9.1 mg (the values are the average of the measurements for three independent scaffolds).

### Example 8—Development of 3D Bioprinted Scaffolds for Probiotic Release to Treat Bacterial Vaginosis

Introduction: Bacterial vaginosis (BV) is a condition in which healthy lactobacilli are replaced by an overabundance of pathogenic bacteria (e.g., Gardenella vaginalis (G.v.)) in the female reproductive tract. Current antibiotic treatments often fail to “cure” the infection, resulting in recurrence in more than 50% of women. One promising approach to treat and potentially prevent recurrent infections is the administration of probiotic organisms, such as lactobacilli, however, there are a dearth of delivery platforms that provide long-term administration (>1 day) of viable probiotics. Three-dimensional (3D)-bioprinting presents a novel approach to intravaginally deliver live probiotic organisms, by fabricating well-defined cell-laden architectures and tuning agent release. A variety of biopolymers can be used as “bioinks,” and gelatin-alginate is a promising candidate for probiotic delivery due to its ability to provide structural stability, host-immune compatibility, viable probiotic incorporation, and nutrient diffusion. In this work, we formulated and characterized novel 3D-bioprinted gelatin-alginate scaffolds to deliver Lactobacillus crispatus (L.cr.) for female reproductive health applications.

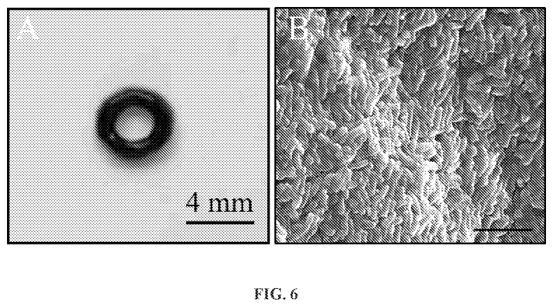

Methods: Different weight to volume (w/v) ratios of gelatin-alginate bioinks (10:2, 11:2, 12:2, 16:4) were bioprinted to determine the formulation with the highest printing resolution and minimal line agglomeration. Additionally, cell-compatible crosslinking reagents including CaCl₂ (10% w/v), genipin (0.5% w/w), and CaCl₂ followed by genipin were evaluated for structural integrity. As proof-of-concept, L.cr.-containing ring scaffolds (OD=4 mm/ID=3 mm, FIG. 6A) were printed, crosslinked and subsequently evaluated for swelling and degradation, post-print probiotic viability, vaginal keratinocyte (VK2/E6E7) cytotoxicity, and sustained-release of probiotics over 4 wk.

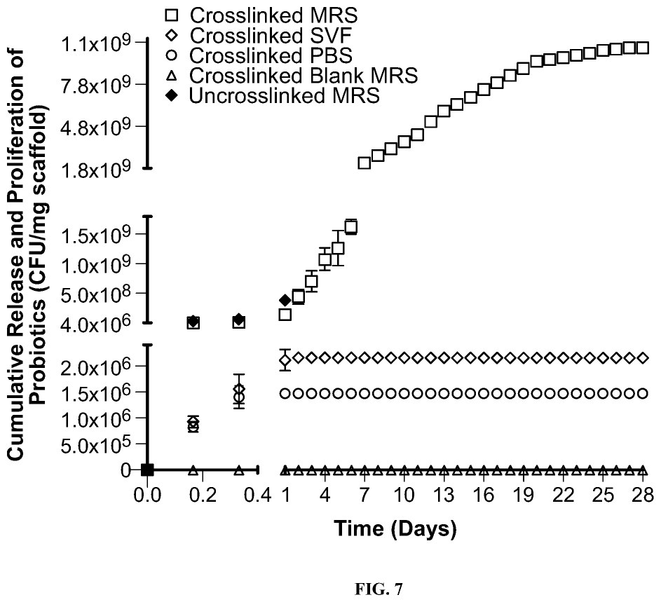

Results: A 10:2 (w/v) bioink formulation was selected based on bioprinted line continuity and resolution (˜600 μm diameter), although none of the ratios failed or were unreadable. Swelling and degradation experiments demonstrated that scaffold crosslinking with both CaCl₂ and genipin produced the greatest structural stability, displaying an intact structure with a total mass loss of only 25% over 28 days and overall swelling of 3.3-fold. Scaffolds incorporating 5×10⁷ CFU L.cr./mg (FIG. 6B) resulted in daily release of ˜4.5×10⁸ CFU/mg (˜9×10⁸ CFU per scaffold), corresponding to cumulative release of ˜10¹⁰ CFUs/mg over 28 days (FIG. 7). Additionally, negligible cytotoxicity was observed in vaginal keratinocytes.

Discussion: 3D-bioprinted L.cr.-containing scaffolds demonstrated high structural integrity and sustained-release of therapeutically-relevant probiotic concentrations over 28 days, with negligible cytotoxicity to vaginal epithelial cells. For the first time, this study demonstrated that 3D-bioprinted scaffolds provides a new and promising alternative to sustain probiotic delivery with future goals to help treat and restore female reproductive health after BV infection.

### Example 9—Development of 3D-Bioprinted Scaffolds Containing Probiotics for Oral Delivery Application

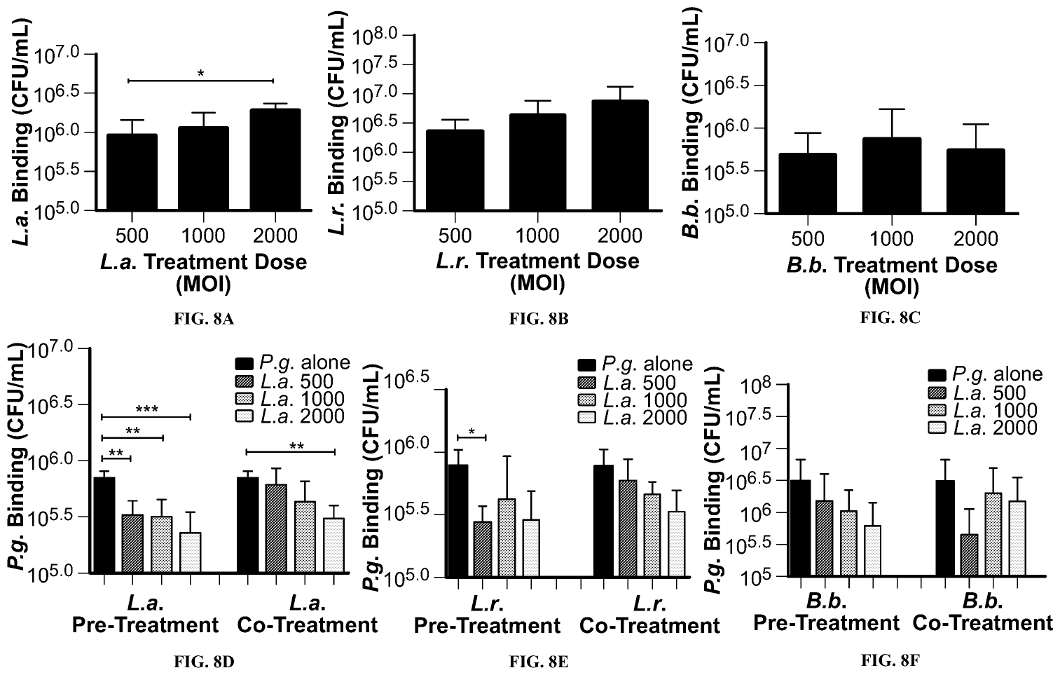

Probiotic binding to TIGK cells was observed after administration of different probiotic MOIs. See FIG. 8A-8C. TIGK cells, plated at a density of 1×10⁵ cells per well, were treated independently with CS-labeled L.a., L.r., or B.b. at MOIs of 500, 1000, 2000 for 90 min. Values representing the mean (±SD) of probiotic binding (CFU/mL) to TIGK cells at increasing MOIs were determined. Increased binding to TIGK cells was only observed between L.a. administered at low (MOI 500) and high concentrations (MOI 2000) (p≤0.05).

Inhibition of P. gingivalis adhesion was observed after different doses of probiotic pre- and co-treatment. See FIG. 8D-8F. L.a., L.r., or B.b. was independently applied to TIGK cells at MOIs of 500, 1000, 2000 for 90 min prior to CS-labeled P. gingivalis administration (MOI 2000) or together with CS-labeled P. gingivalis (MOI 2000)-treated for 90 min. Values representing the mean (±SD) of P. gingivalis adhesion (CFU/mL) to TIGK cells in the absence (P. gingivalis alone) or presence of L.a. pre-or co-treatment were determined. Statistical significance between P. gingivalis alone and probiotic-treated groups was calculated by one-way ANOVA and is represented by *P≤0.05. **P≤0.01, and ***P≤0.001.

After pre-treatment, L.a. administered at all doses significantly inhibited P. gingivalis binding to TIGK cells. For L.a. and P. gingivalis co-treatment, P. gingivalis inhibition was observed at the highest dose of L.a. (2000 MOI). No statistical significance was observed between P. gingivalis alone and L.r. pre- or co-treated groups, with the exception of P. gingivalis and L.r. (500) pre-treatment. Both P.g. pre- and co-treatment with B.b., showed similar P. gingivalis binding compared to the untreated control group (P>0.05). Overall, probiotic pre-treatment resulted in a slightly higher reduction in P. gingivalis binding, relative to that observed for the same co-treatment doses (P>0.05).

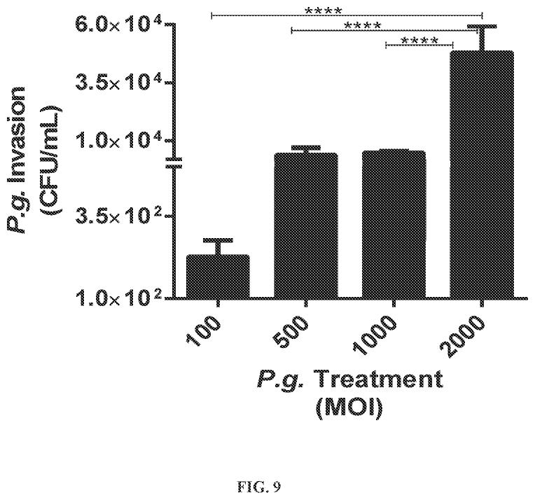

In addition, TIGK cells showed a dose-dependent increase in P. gingivalis internalization (FIG. 9). Values representing the mean (±SD) of P. gingivalis in CFU/mL were determined. Statistical significance in internalization as a function of administered P.g. dose was calculated by one-way ANOVA and is represented in FIG. 9 by ****P≤0.0001. FIG. 9 shows that, at an MOI of 2000, P.g. internalization was significantly higher relative to P.g. administered at MOIs of 100, 500, 1000.

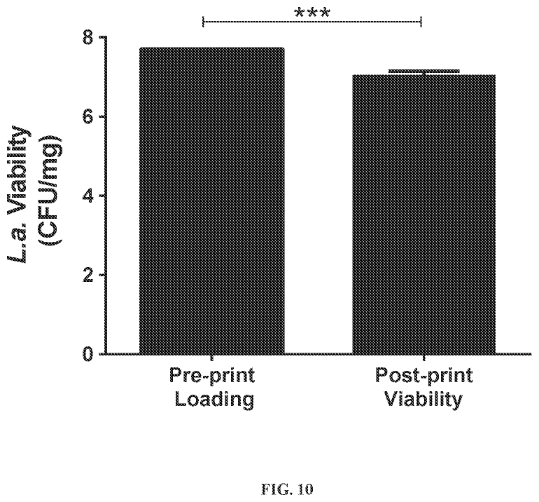

Probiotic viability also was determined pre- and post-printing after initial incorporation of 5×10⁷ CFU L.a. per mg scaffold (FIG. 10). Values representing the mean (±SD) of post-print viability were determined. Statistical significance was calculated by t-test and is represented in FIG. 10 by ***P≤0.001.

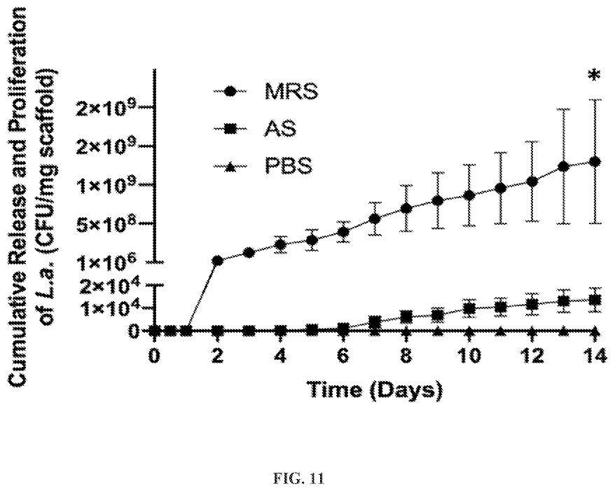

Cumulative release of L.a. from 3D-bioprinted scaffolds in PBS, MRS, and artificial saliva was examined (FIG. 11). Values representing the mean (±SD) of cumulative release at each time point were determined. Statistical significance was calculated by t-test and is represented in FIG. 11 by p≤0.05. This data was obtained in collaboration with Veeresh Rai.

All probiotics demonstrated adhesion to TIGK cells. Among the three probiotics examined, L.a. demonstrated a statistically significant reduction in P. gingivalis binding after pre-treatment at all MOIs. Adhesion reduction was significant for L.a. co-treatment group only at the highest MOI.

Assessment of P. gingivalis internalization with the antibiotic protection assay indicated that P. gingivalis invasion of TIGK cells exhibits some level of dose-dependence. Invasion increased as a function of the dose. For example, P. gingivalis administered at an MOI of 2000 showed statistically significant levels of invasion relative to lower MOIs.

Probiotics were highly viable in 3D bioprinted scaffolds (˜10⁷) and sustained release was demonstrated in MRS and artificial saliva for up to 14 days.

### Example 10—Development of 3D-Bioprinted Scaffolds for Probiotic Release to Treat Bacterial Vaginosis

3D-bioprinting offers a long-term, novel approach to intravaginally sustain the delivery of live probiotics, to prevent/treat initial infection, and/or reduce recurrent BV infections. Experiments were performed to fabricate and characterize 3D-bioprinted probiotic-containing scaffolds. The goal was to generate scaffolds that sustain the release of probiotics for a minimum duration of one week. Specifically, the goal is to intravaginally deliver live probiotic organisms (e.g., L.cr.) by fabricating and characterizing probiotic architectures that modulate release.

Methods. Methods were used to generate and examine multiple facets of a 3D scaffold. For example, the optimal ratio of gelatin-alginate bioink, the optimization of printing parameters, printing resolution, probiotic (L.cr.) viability, probiotic release and proliferation, mass loss and swelling, and cytotoxicity to vaginal epithelial cells.

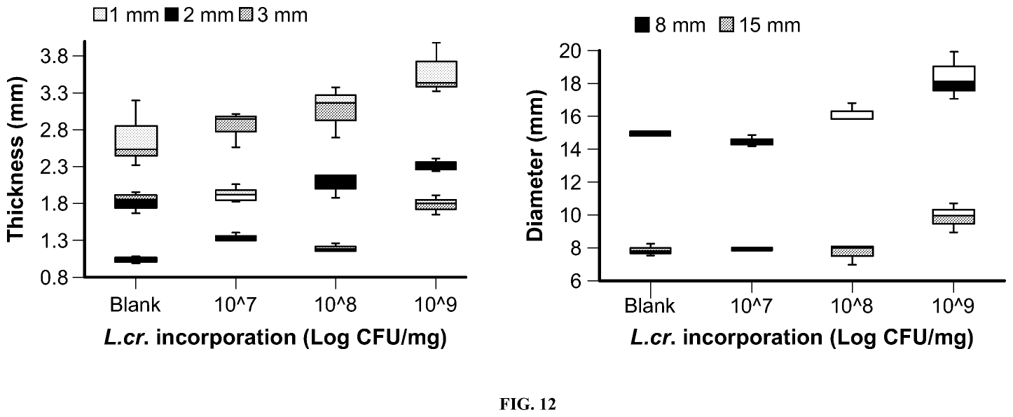

The results of the experiments related to gelatin-alginate print resolution are shown in FIG. 12.

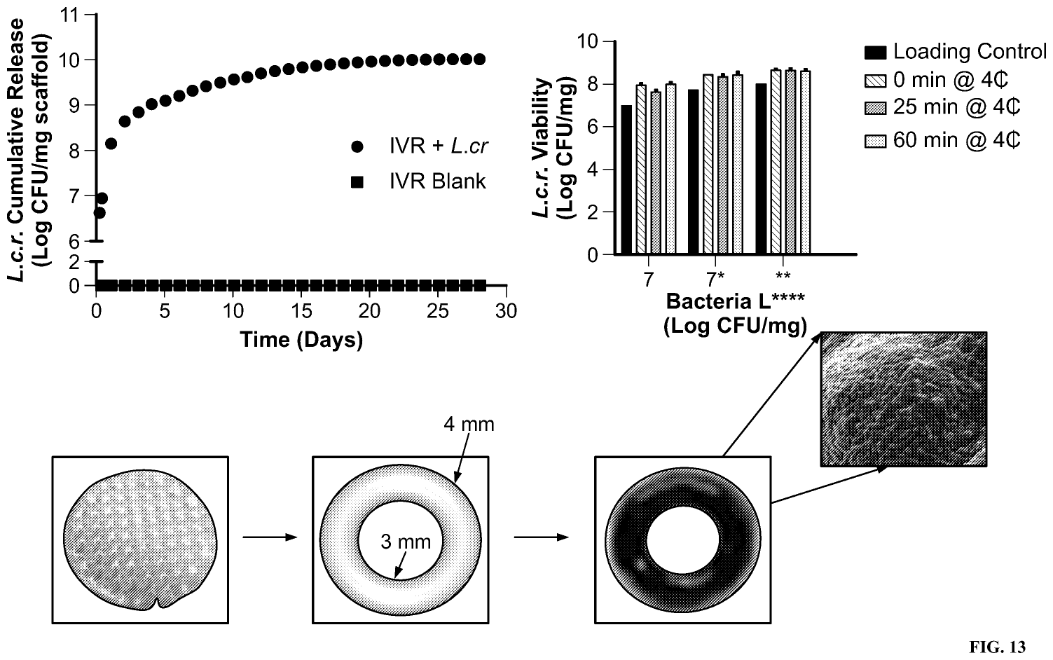

The results of the experiments related to post-print viability and probiotic release/proliferation are shown in FIG. 13. A crosslinked ring is advantageous for, without limitation, mechanical integrity, enables sustained-release delivery, and may provide an environment for cell proliferation. Based on these experiments, L.cr. viability was shown to be most advantageous at 5×10⁷ CFU/mg, whereas proliferation plateaued at day 14 with 5.0×10⁹ CFU/mg.

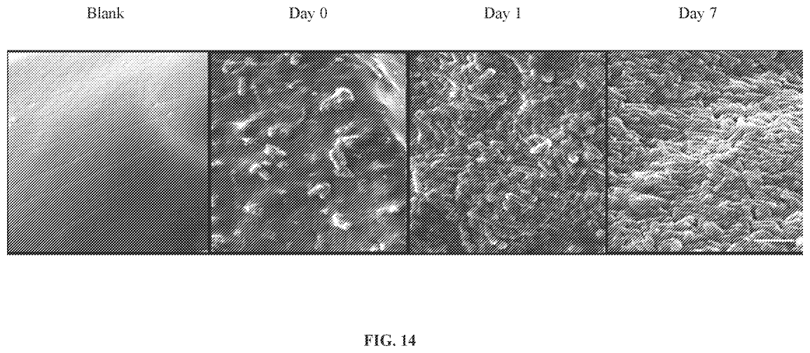

SEM images of scaffold cross-sections were taken at various time points to examine proliferation and morphology of L. cr.-containing scaffolds (see FIG. 14). Proliferation of probiotics within the scaffold was observed between Day 1 and Day 7.

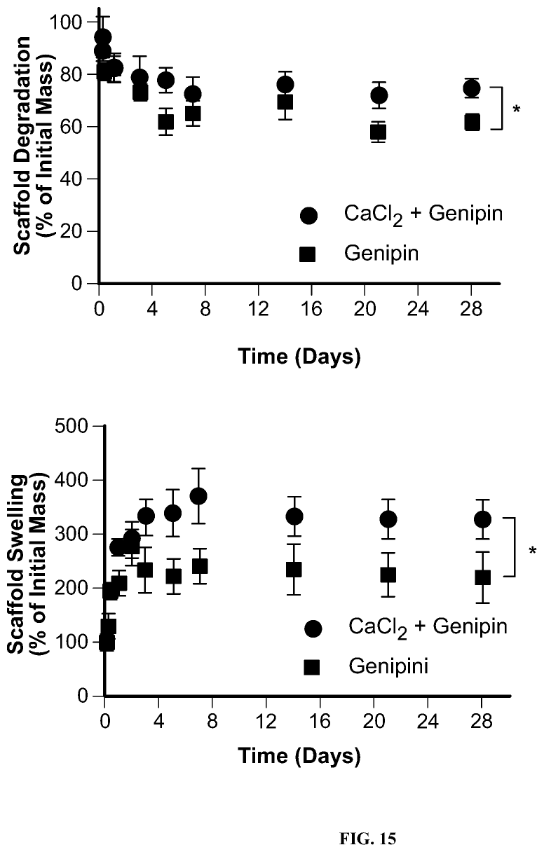

Scaffolds were evaluated for swelling and degradation across different crosslinking parameters corresponding to release studies. Both CaCl₂+Genipin and Genipin-only scaffolds demonstrated stable degradation and swelling profiles after ˜4 days. For example, CaCl₂+Genipin exhibited a total mass loss of only 25% and a swelling of only 3.3-fold over 28 days. See FIG. 15.

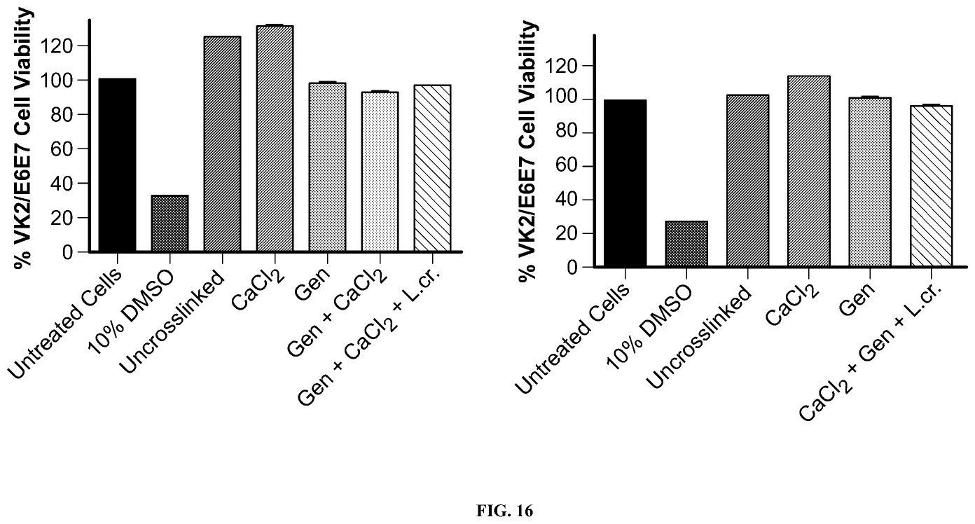

Uncrosslinked scaffolds and scaffolds with different crosslinkers were exposed for 24 and 72 hr to vaginal keratinocytes (VK2/E6E7), and cell viability was determined using an MTT assay. Negligible cytotoxicity was observed in treated keratinocytes; toxicity was comparable to untreated cells (FIG. 16).

Data from this study demonstrated that 3D-bioprinted L.cr.-containing scaffolds can be fabricated to provide fine printing resolution; high probiotic loading; sustained probiotic release and proliferation; stable mass loss and degradation after 4 days; and biocompatibility with vaginal epithelial cells. For the first time, experiments demonstrated that 3D-bioprinted scaffolds can provide a new alternative for sustained probiotic delivery.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A method of making a probiotic delivery system, comprising the steps of:
combining at least one probiotic with bioink to produce a probiotic-seeded bioink; and
printing a three-dimensional structure using the probiotic-seeded bioink.

2. The method of claim 1, wherein the at least one probiotic is selected from a genus consisting of Lactobacillus, Bifidobacterium, and combinations thereof.

3. The method of claim 1, wherein the at least one probiotic is selected from the group consisting of L. acidophilus, L. crispatus, L. rhamnosus, L. gasseri, L. reuteri, L. bulgaricus, L. plantarum, L. johnsonii, L. paracasei, L. casei, and L. salivaris.

4. The method of claim 1, wherein the at least one probiotic is selected from the group consisting of B. bifidum, B. longum, B. breve, B. infantis, B. lactis, and B. adolescentis.

5. The method of claim 1, wherein between about 10\hat{ }6 and 10\hat{ }9 cfu per mg polymer of the at least one probiotic is combined with the bioink.

6. The method of claim 1 any of the preceding claims, wherein between about 10\hat{ }6 and 10\hat{ }9 cfu of the at least one probiotic per mg polymer is combined with the bioink.

7. The method of claim 1, wherein the bioink comprises agarose, alginate, chitosan, collagen, decellularized extracellular matrix (ECM), fibrin/fibrinogen, gelatin, graphene, hyaluronic acid (HA), hydroxyapatite, PCL/PLA/PLGA, silicone, Pluronic F127, polyethylene glycol/oxide, and combinations thereof.

8. The method of claim 1, wherein the bioink comprises alginate and gelatin.

9. The method of claim 8, wherein the bioink comprises about 2% w/v alginate and about 10% w/v gelatin.

10. The method of claim 1, wherein the printing comprises extrusion, co-axial extrusion, fused deposition modelling, inkjet bio-printing, laser-assisted bioprinting, stereolithography, selective laser sintering (SLS), or combinations thereof.

11. The method of claim 1, wherein the structure is line-shaped, cylindrical-shaped, disc-shaped, spherical-shaped, oval-shaped, cube-shaped, layered, a structure with discreet compartment, or combinations thereof.

12. The method of claim 1, further comprising crosslinking the structure.

13. The method of claim 12, wherein the crosslinking comprises applying genipin, calcium chloride, gelatin methacryloyl, ultraviolet (UV) light, or combinations thereof.

14. A probiotic delivery system made by the method of claim.

15. A probiotic delivery system comprising a bioink and at least one probiotic.

16. The system of claim 15, wherein the bioink comprises sodium alginate and gelatin.

17. The system of claim 16, wherein the bioink comprises about 2% w/v sodium alginate and about 10% w/v gelatin.

18. The system of claim 15, wherein the at least one probiotic is selected from a genus consisting of Lactobacillus, Bifidobacterium, and combinations thereof.

19. A method of treating a bacterial infection in an individual, comprising:
administering the probiotic delivery system of claim 15 to the individual.

20. A method of treating a bacterial infection in an individual, comprising:
printing the probiotic delivery system of claim 15; and
administering the printed probiotic delivery system to the individual.

21. The method of claim 20, wherein the bacterial infection is caused by the presence of at least Porphyromonas gingivalis or Gardnerella vaginalis.
